SEQUENCE LISTING (19 sequences)

SEQ ID NO: 1 - DNA, 20 bp, Artificial sequence
  P1 site forward primer
ggggaaggaacttacatgac

SEQ ID NO: 2 - DNA, 20 bp, Artificial sequence
  P1 site reverse primer
tcgtgtttccctgttgtacc

SEQ ID NO: 3 - DNA, 20 bp, Artificial sequence
  PA site forward primer
ggtctaagccacacactaac

SEQ ID NO: 4 - DNA, 20 bp, Artificial sequence
  PA site reverse primer
tgagtttagggaccaatcta

SEQ ID NO: 5 - DNA, 21 bp, Artificial sequence
  PB site forward primer
gctgccagtatgtgaccgaga

SEQ ID NO: 6 - DNA, 20 bp, Artificial sequence
  PB site reverse primer
gggtggaggagaggataggg

SEQ ID NO: 7 - DNA, 25 bp, Artificial sequence
  T7 reverse primer
tcgccctatagtgagtcgtattaca

SEQ ID NO: 8 - DNA, 25 bp, Artificial sequence
  SP6 reverse primer
gtattctatagtgtcacctaaatag

SEQ ID NO: 9 - DNA, 41 bp, Artificial sequence
  CS reverse primer
gactcactatagggcgaggggaagggagcgggaggtttgtg

SEQ ID NO: 10 - DNA, 44 bp, Artificial sequence
  CS reverse primer
ggtgacactatagaatacgcgctgagcctgccgggaagtgtagt

SEQ ID NO: 11 - DNA, 19 bp, Artificial sequence
  TBX6 primer
ctcgaaggggtccgagagg

SEQ ID NO: 12 - DNA, 20 bp, Artificial sequence
  TBX6 primer
ctccttccatagctcccggt

SEQ ID NO: 13 - DNA, 20 bp, Artificial sequence
  TBX6 primer
gttgcatactgatcccgaat

SEQ ID NO: 14 - DNA, 20 bp, Artificial sequence
  TBX6 primer
ctgcccgaactaggtgtatg

SEQ ID NO: 15 - DNA, 21 bp, Artificial sequence
  TBX6 primer
aatggcttcctaacagatgac

SEQ ID NO: 16 - DNA, 19 bp, Artificial sequence
  TBX6 primer
gagcgggaggtttgtgatg

SEQ ID NO: 17 - DNA, 19 bp, Artificial sequence
  TBX6 primer
ggcagctggaaacacaggt

SEQ ID NO: 18 - DNA, 25 bp, Artificial sequence
  TBX6 forward primer
tagggagagggctctgttctcatgg

SEQ ID NO: 19 - DNA, 20 bp, Artificial sequence
  TBX6 reverse primer
gcgtcccagggaggcaaccg

(19) United States
(12) Patent Application Publication
(10) Pub. No.: US 2019/0376141 A1
(43) Pub. Date: Dec. 12, 2019

(54) Product for Diagnosing Congenital Scoliosis and Application Thereof

(71) Applicants: Nan Wu, Beijing (CN); Zhihong Wu, Beijing (CN); Guixing Qiu, Beijing (CN)

(72) Inventors: Nan Wu, Beijing (CN); Zhihong Wu, Beijing (CN); Guixing Qiu, Beijing (CN)

(21) Appl. No.: 16/552,398

(22) Filed: Aug. 27, 2019

Related U.S. Application Data
(63) Continuation of application No. 15/321,377, filed on Apr. 21, 2017, now abandoned.

(30) Foreign Application Priority Data
Jun. 24, 2014 (CN) 201410284657.5
Oct. 24, 2014 (CN) 201410572962.4

(51) Int. Cl. C12Q 1/6883

(52) U.S. Cl. CPC C12Q 1/6883; C12Q 2600/16; C12Q 2600/158; C12Q 2600/156

(57) ABSTRACT

The present invention discloses a method for diagnosing congenital scoliosis of an individual, comprising: detecting whether a chromosome 16p11.2 region has a nucleotide sequence microdeletion of 0.6 Mb in length, or detecting whether a TBX6 gene has a frameshift mutation; and detecting a haplotype of two SNP sites of rs3809624-rs3809627 in the TBX6 gene located on another homologous chromosome. The diagnostic method of the present invention can be judged early in the congenital scoliosis and is suitable for clinical promotion.

### CROSS-REFERENCE TO RELATED APPLICATIONS

This application is a continuation of U.S. patent application Ser. No. 15/321,377, filed Apr. 21, 2017, which is the United States national phase of International Application No. PCT/CN2015/076692 filed Apr. 16, 2015, and claims priority to Chinese Patent Application Nos. 201410284657.5 and 201410572962.4 filed Jun. 24, 2014, and Oct. 24, 2014, respectively, the disclosures of which are hereby incorporated in their entirety by reference.

The Sequence Listing associated with this application is filed in electronic format via EFS-Web and is hereby incorporated by reference into the specification in its entirety. The name of the text file containing the Sequence Listing is 1606352_ST25.txt. The size of the text file is 4,018 bytes, and the text file was created on Mar. 8, 2017.

### TECHNICAL FIELD

The present application belongs to the field of medical diagnosis, which relates to a method and product for diagnosing disease, and particularly relates to a method and kit for diagnosing congenital scoliosis.

### BACKGROUND ART

Array-comparative genomic hybridization (Array-CGH) is a high-throughput analytical technology developed on the basis of traditional CGH. With this technology, BAC cloned DNA or oligonucleotides are made into a microarray for replacing metaphase chromosomes taken as a hybridization target for detection in the traditional CGH detection, which not only improves resolution but also provides precise localization. Meanwhile, each chromosome can be recognized by computer software, which overcomes the limitation that experienced staff are required for chromosome identification, thus providing a relatively ideal method for the rapid and comprehensive analysis of changes in DNA copy numbers and the instability detection of chromosomes. In this technology, after the genomic DNA is extracted from tissue cells, non-specific repetitive sequences of same amount of different fluorescently labeled samples and reference DNA are sealed by human Cot-1 DNA, and the labeled samples and reference DNA are hybridized simultaneously to the microarray composed of DNA or oligonucleotides. Changes in the copy numbers of the genomic DNA to be detected in corresponding sequences or genes are reflected by the fluorescence ratio of two types of signals on each target spot of the microarray. Since the first article about utilizing the gene chip technology to study gene levels issued in the journal Science by Schena et al. from Stanford University in 1995, this technology has been applied to the screening of genes and diagnostic indicators with respect to the occurrence and development of various congenitally handicapped and neoplastic diseases, especially has been applied to the studies on molecular subtyping of various malignant tumors and prediction of therapeutic response, tumors metastasis and recurrence and prognosis, and great achievements have been made. The main contents of the study include changes in tumor gene copy number, specific gene region analysis and a series of clinical application related research, such as assisting in pathological type diagnosis, screening tumor prognosis related markers and so on.

Congenital scoliosis (CS) is a common spinal disease, the neonatal morbidity is 0.5-1‰. The clinical manifestation of CS is a scoliosis of more than 10 degrees, which is due to the spinal longitudinal growth imbalance caused by the spinal deformity (such as hemivertebrae deformity, segmental disorder, butterfly vertebrae, fused ribs, etc.) during the embryonic development process. CS can affect the physical and mental health, and has become a major factor in adolescent disability.

In the past, it is believed that most of the congenital scoliosis is not hereditary but caused by environmental factors in the development process of the embryo. In recent years, studies show that genetic factors are involved in the pathogenesis of CS. Previous genetic manipulation experiments in animal models show that genetic defects lead to spinal abnormalities. Interestingly, it is shown that some mutations in human genes (e.g., DLL3, HEST, MESP2 and 7) are involved in the CS process; however, these mutations can be inherited from phenotypically normal family members. Phenotypic differences caused by identical mutations within the family are indicative of the complexities of human CS genetic variation. Interactions between genes and the environment are proposed to explain the above phenomena.

Several studies have reported that patients with a human chromosome 16p11.2 microdeletion have a CS phenotype, so we hypothesize that genetic modifying factors and cofactors may be another mechanism for CS genetic variation. About 0.6 Mb deletion of human chromosome 16p11.2 is a rare human mutation, the mutation frequency is about 0.02%. This deletion can cause neurodevelopmental related diseases (e.g., autism and obesity). Interestingly, the phenotype of CS has recently been found in a small number of patients with the 16p11.2 microdeletions, which suggests that 16p11.2 microdeletion may be involved in the pathogenesis of CS. In addition, the low penetrance of 16p11.2 microdeletion in CS also highlights the complexity of the human CS genetic mechanism.

Although CS is often found in infants or early childhood, it is not manifested until their adolescence for many children. Due to the lack of diagnostic knowledge and diagnostic means and other reasons, the lesion is often ignored by parents and doctors, and not found until after the obvious development of deformity. Now, the usual methods of diagnosing congenital scoliosis include X-ray, MRI, and so on, but these methods are only applicable to patients with obvious lesions, and not applicable to those potential patients who have not yet an obvious phenotype, therefore, the development of a sensitive method capable of diagnosing the congenital scoliosis in early stage is a problem need to be solved.

### SUMMARY OF THE INVENTION

The first object of the present application is to provide a method for diagnosing congenital scoliosis, the method of the present application is suitable for early diagnosis of congenital scoliosis.

The second object of the present application is to provide a product for diagnosing congenital scoliosis, and compared with the detection by using medical instruments traditionally, the detection by using the product is more sensitive, and is suitable for the early diagnosis of congenital scoliosis.

In order to achieve the above object, the present application adopts the following technical solutions:

The present application provides a method for diagnosing congenital scoliosis, the method comprises determining whether a chromosome 16p11.2 region has a mutation, and based on the mutation, determining whether the subject has congenital scoliosis.

Mutations in the chromosome 16p11.2 region include nucleotide deletions, nucleotide insertions, and nucleotide mutations. The nucleotide microdeletion in the chromosome 16p11.2 region is a deletion of a nucleotide sequence with a length of 0.6 Mb in the chromosome 16p11.2 region, referred to as a 16p11.2 microdeletion.

In a specific embodiment of the present application, the present application provides a method of diagnosing congenital scoliosis of an individual. The method includes detecting whether a chromosome 16p11.2 region has a nucleotide sequence microdeletion of 0.6 Mb in length, and detecting a haplotype of two SNP sites of rs3809624-rs3809627 in the TBX6 gene located on another homologous chromosome.

If a microdeletion of 0.6 Mb in length exists in the chromosome 16p11.2 region, meanwhile the haplotype of two SNP sites of rs3809624-rs3809627 in the TBX6 gene located on the another homologous chromosome without 16p11.2 microdeletion is C-A, the individual is diagnosed as a patient with congenital scoliosis.

Further, the method includes: obtaining a biological sample containing the genomic DNA of the subject; and extracting genomic DNA in the biological sample.

The above method comprises the use of QPCR, high-density oligonucleotide comparative genomic hybridization microarray or sequencing technology to detect whether a microdeletion of 0.6 Mb in length exists in the chromosome 16p11.2 region; and the detection of the haplotype of two SNP sites of rs3809624-rs3809627 in the TBX6 gene is implemented by sequencing.

The use of QPCR to detect whether a microdeletion of 0.6 Mb in length exists in the chromosome 16p11.2 region is implemented by the use of primers for amplifying a nucleotide sequence having a length of 0.6 Mb located between 29.5 Mb and 30.1 Mb in the chromosome 16p11.2 region, and the sequences of the primers are as follows: P1 site forward primer 5′-GGGGAAGGAACTTACATGAC-3′ (SEQ ID NO: 1), P1 site reverse primer 5′-TCGTGTTTCCCTGTTGTACC-3′ (SEQ ID NO: 2), PA site forward primer 5′-GGTCTAAGCCACACACTAAC-3′(SEQ ID NO: 3), PA site reverse primer 5′-TGAGTTTAGGGACCAATCTA-3′ (SEQ ID NO: 4), PB site forward primer 5′-GCTGCCAGTATGTGACCGAGA-3′ (SEQ ID NO: 5), PB site reverse primer 5′-GGGTGGAGGAGAGGATAGGG-3′ (SEQ ID NO: 6).

The specific protocol for QPCR is to select two detection sites of PA and PB in the 16p11.2 microdeletion region, select a reference site P1 outside the 16p11.2 microdeletion region, and design primers using P1 and PA or P1 and PB combinations to amplify different fragments, and the amount of fragments present is detected by a conventional QPCR method. In a specific embodiment, the above primer sequences are as follows: P1 site forward primer5′-GGGGAAGGAACTTACATGAC-3′ (SEQ ID NO: 1), P1 site reverse primer 5′-TCGTGTTTCCCTGTTGTACC-3′ (SEQ ID NO: 2), PA site forward primer 5′-GGTCTAAGCCACACACTAAC-3′(SEQ ID NO: 3), PA site reverse primer 5′-TGAGTTTAGGGACCAATCTA-3′(SEQ ID NO: 4), PB site forward primer 5′-GCTGCCAGTATGTGACCGAGA-3′ (SEQ ID NO: 5), PB site reverse primer 5′-GGGTGGAGGAGAGGATAGGG-3′ (SEQ ID NO: 6).

In a specific embodiment of the present application, the present application provides a method of diagnosing congenital scoliosis of an individual. The method includes: detecting whether a TBX6 gene has a frameshift mutation, and detecting a haplotype of two SNP sites of rs3809624-rs3809627 in the TBX6 gene located on another homologous chromosome.

If the TBX6 gene in the chromosome 16p11.2 region has the following single nucleotide insertion and double nucleotide deletion: one or more of nucleotide shift mutations caused by c.1248-1249insT, c.263-264insC, c.697-698insG, c.1167-1168insC, c.1179-1180delAG, and the haplotype of two SNP sites of rs3809624-rs3809627 in the TBX6 gene located on the homologous chromosome without frameshift mutations is C-A, then the individual is diagnosed as a patient with congenital scoliosis.

Further, the method includes: obtaining a biological sample containing the genomic DNA of the subject; and extracting genomic DNA in the biological sample.

Further, the method includes: detecting whether a TBX6 gene has a frameshift mutation, and simultaneous detecting a haplotype of two SNP sites of rs3809624-rs3809627 in the TBX6 gene located on another homologous chromosome;

If the TBX6 gene in the chromosome 16p11.2 region has the following single nucleotide insertion and double nucleotide deletion: one or more of nucleotide shift mutations caused by c.1248-1249insT, c.263-264insC, c.697-698insG, c.1167-1168insC, c.1179-1180delAG, and the haplotype of two SNP sites of rs3809624-rs3809627 in the TBX6 gene located on the homologous chromosome without frameshift mutations is C-A, then the individual is diagnosed as a patient with congenital scoliosis.

The above method includes the use of sequencing technology to detect whether a TBX6 gene has a frameshift mutation; the use of sequencing technology to detect whether a TBX6 gene has a frameshift mutation requires amplification primers and sequencing primers, the amplification primers are as follows: forward primer 5′-TAGGGAGAGGGCTCTGTTCTCATGG-3′(SEQ ID NO: 18); reverse primer 5′-GCGTCCCAGGGAGGCAACCG-3′ (SEQ ID NO: 19); the sequencing primers are as follows: 5′-CTCGAAGGGGTCCGAGAGG-3′ (SEQ ID NO: 11), 5′-CTCCTTCCATAGCTCCCGGT-3′(SEQ ID NO: 12), 5′-GTTGCATACTGATCCCGAAT-3′(SEQ ID NO: 13), 5′-CTGCCCGAACTAGGTGTATG-3′(SEQ ID NO: 14), 5′-AATGGCTTCCTAACAGATGAC-3′, 5′-GAGCGGGAGGTTTGTGATG-3′ (SEQ ID NO: 16), 5′-GGCAGCTGGAAACACAGGT-3′(SEQ ID NO: 17).

The method for detecting whether the TBX6 gene has a frameshift mutation by using sequencing technology comprises: (1) amplification of the full-length TBX6 gene; (2) sanger sequencing.

Further, the method for detecting whether the TBX6 gene has a frameshift mutation by using sequencing technology comprises: (1) designing reasonable primers for amplifying a TBX6 gene coding region and an upstream regulatory region of nearly 1kb; (2) sequencing the amplified fragments in step (1) by using sequencing primers. Preferably, the sequences of the amplification primers are as follows: forward primer 5′-TAGGGAGAGGGCTCTGTTCTCATGG-3′ (SEQ ID NO: 18); reverse primer 5′-GCGTCCCAGGGAGGCAACCG-3′ (SEQ ID NO: 19). The sequences of the sequencing primers are as follows: 5′-CTCGAAGGGGTCCGAGAGG-3′ (SEQ ID NO: 11), 5′-CTCCTTCCATAGCTCCCGGT-3′ (SEQ ID NO: 12), 5′-GTTGCATACTGATCCCGAAT-3′ (SEQ ID NO: 13), 5′-CTGCCCGAACTAGGTGTATG-3′ (SEQ ID NO: 14), 5′-AATGGCTTCCTAACAGATGAC-3′ (SEQ ID NO: 15), 5′-GAGCGGGAGGTTTGTGATG-3′ (SEQ ID NO: 16), 5′-GGCAGCTGGAAACACAGGT-3′ (SEQ ID NO: 17).

The method for detecting the genotypes of the rs3809624 site and the rs3809627 site in the TBX6 gene by using sequencing technology comprises: (1) amplifying a vector and inserted DNA fragments, connecting and constructing a recombinant vector of the TBX6 gene; (2) transforming the recombinant vector into competent cells of Escherichia coli; (3) selecting clones, designing sequencing primers and detecting sequences by sanger sequencing. Preferably, the vector is pGEM-T. The sequences of the primers required for constructing the recombinant vector of the TBX6 gene are as follows: T7 reverse primer 5′-TCGCCCTATAGTGAGTCGTATTACA-3′ (SEQ ID NO: 7), SP6 reverse primer 5′-GTATTCTATAGTGTCACCTAAATAG-3′ (SEQ ID NO: 8), CS forward primer 5′-GACTCACTATAGGGCGAGGGGAAGGGAGCGGGAGGTTTGTG-3′ (SEQ ID NO: 9), CS reverse primer 5′-GGTGACACTATAGAATACGCGCTGAGCCTGCCGGGAAGTGTAGT-3′ (SEQ ID NO: 10). Preferably, the sequences of the sequencing primers are as follows: 5′-CTCGAAGGGGTCCGAGAGG-3′ (SEQ ID NO: 11), 5′-CTCCTTCCATAGCTCCCGGT-3′ (SEQ ID NO: 12), 5′-GTTGCATACTGATCCCGAAT-3′ (SEQ ID NO: 13), 5′-CTGCCCGAACTAGGTGTATG-3 (SEQ ID NO: 14)', 5′-AATGGCTTCCTAACAGATGAC-3′ (SEQ ID NO: 15), 5′-GAGCGGGAGGTTTGTGATG-3′ (SEQ ID NO: 16), 5′-GGCAGCTGGAAACACAGGT-3′ (SEQ ID NO: 17).

The biological sample of the present application includes, but is not limited to, tissue, and body fluids.

Preferably, the biological sample is a body fluid. The body fluid includes, but is not limited to, blood, plasma, saliva, urine, and amniotic fluid.

More preferably, the biological sample is blood.

In a specific embodiment of the present application, extracting genomic DNA from a biological sample refers to extracting genomic DNA from peripheral blood leukocytes. Extraction of the detected genomic DNA is carried out according to techniques well known to those skilled in the art.

In a specific embodiment of the present application, a frameshift mutation caused by nucleotide insertion occurs in the TBX6 gene. TBX6 gene expression amount is reduced when the frameshift mutation occurs, and of course presence of other frameshift mutations which also affect TBX6 gene expression is not excluded.

Experiments in the present application prove that TBX6 gene expression is down-regulated when the haplotype of rs3809624-rs3809627 sites is C-A. Detailed operation steps for the demonstration are: (1) amplifying a 1120bp DNA fragment of an upstream regulatory element of the TBX6 gene, and constructing a normal DNA fragment, a DNA fragment with only rs3809624 site mutated to C, a DNA fragment with only rs3809627 site mutated to A, a DNA fragment with rs3809624 site and rs3809627 site mutated at the same time onto a pGL3-Basic vector respectively; (2) transfecting recombinant vectors into HEK293T, HepG2, Hela cells cultured in vitro; (3) after transfection for a certain period of time, lysing the cells and obtaining supernatants to detect the activity of luciferase by using a Dual-Luciferase Reporter Gene Assay system.

In a specific embodiment of the present application, human cells are HEK293T, HepG2, and Hela. Preferably, the vector with a luciferase reporter gene used in the present application is pGL3-Basic vector, and a control vector is pRL-TK.

By utilizing QPCR and a high-density oligonucleotide comparative genomic hybridization technique, the present disclosure identifies 12 individuals with the chromosome 16p11.2 microdeletion from a population of 161 patients who are not related to each other and suffer from congenital scoliosis. Four individuals with the TBX6 gene frameshift mutation caused by single nucleotide insertion are simultaneously identified from the above population of patients by utilizing a DNA sequencing technique. Four cases of the single nucleotide insertion are: c.1248-1249insT, c.263-264insC, c.697-698insG, c.1167-1168insC. The Chromosome deletion and the frameshift mutation are collectively referred to as nonsense mutations. 16 individuals from the population of 161 patients are identified to have a nonsense mutation, and the probability of the nonsense mutation in CS is 10.6%. By the same techniques, the present application re-collects a population of 76 patients who are not related to each other and suffer from congenital scoliosis, and 5 individuals with the chromosome 16p11.2 microdeletion are identified. One individual with the TBX6 gene frameshift mutation caused by double nucleotide deletions is simultaneously identified from the above population. The case of double nucleotide deletions is: c.1179-1180delAG. 6 individuals from the population of 76 patients are identified to have the nonsense mutation, and the probability of the nonsense mutation in CS is 7.9%. Combined with the results of the two experiments, it is found that the probability of the nonsense mutation in CS is between 7.9%-10.6%.

Two families with the chromosome 16p11.2 microdeletion are selected as objects of study, and the phenotype of each individual is analyzed. Although father or siblings of an individual with CS have a 16p11.2 microdeletion, they do not have the CS phenotype. It has been hypothesized that other factors assist and involve in the manifestation of the CS phenotype. When analyzing the haplotype of the TBX6 gene located on the homologous chromosome without the 16p11.2 microdeletion, it is found that when the haplotype of three SNP sites of rs2289292-rs3809624-rs3809627 is T-C-A, the individual with the chromosome 16p11.2 microdeletion exhibits CS, otherwise the individual does not exhibit CS even having the chromosome 16p11.2 microdeletion. When simultaneously detecting the haplotypes of three SNP sites of rs2289292-rs3809624-rs3809627 in the TBX6 gene located on another normal chromosome of the 16 individuals with nonsense mutations identified from the population of 161 patients and the 6 individuals with nonsense mutations identified from the population of 76 patients, it is found that haplotypes of the three SNP sites all are T-C-A. The mutation of rs2289292 of the three SNP sites does not alter protein encoding of the TBX6 gene. And rs3809624 and rs3809627 locate within the upstream regulatory sequence of the TBX6 gene, effects of the mutations of the rs3809624 and rs3809627 sites on gene expression are tested by a luciferase reporter system, and the results show that gene expression is inhibited when both rs3809624 and rs3809627 sites mutate. Since mutation at rs2289292 site does not affect the protein encoding of the TBX6 gene, and it has been reported that rs2289292 site and rs3809624 site are in a linkage disequilibrium region, the haplotype of rs2289292 site is T when the haplotype of the rs3809624 and rs3809627 sites is determined to be C-A.

Accordingly, the present disclosure discloses criteria for judging congenital scoliosis:

(1) when a chromosome 16p11.2 microdeletion exists, and the haplotype of two SNP sites of rs3809624-rs3809627 in the TBX6 gene located on the homologous chromosome without the 16p11.2 microdeletion is C-A, an individual is diagnosed as a patient with congenital scoliosis.

(2) when one or more of nucleotide frameshift mutations caused by the following single nucleotide insertions and a double nucleotide deletion: c.1248-1249insT, c.263-264insC, c.697-698insG, c.1167-1168insC, c.1179-1180delAG exist in the TBX6 gene within the chromosome 16p11.2 region, and the haplotype of two SNP sites of rs3809624-rs3809627 in the TBX6 gene located on the homologous chromosome without frameshift mutations is C-A, an individual is diagnosed as a patient with congenital scoliosis.

Based on the above theory, the present application develops a diagnostic kit for congenital scoliosis, comprising: an agent for determining whether a chromosome 16p11.2 region has a nucleotide sequence microdeletion of 0.6 Mb in length, or determining whether a TBX6 gene has a frameshift mutation, and determining the haplotype of two SNP sites of rs3809624-rs3809627 in the TBX6 gene located on another homologous chromosome;

The frameshift mutation of the TBX6 gene is selected from the following single nucleotide insertions and dinucleotide deletions: one or more of nucleotide shift mutations caused by c.1248-1249insT, c.263-264insC, c.697-698insG, c.1167-1168insC, c.1179-1180delAG.

The reagents for determining whether a chromosome 16p11.2 region has a nucleotide sequence microdeletion of 0.6 Mb in length include the reagents used in QPCR, high-density oligonucleotide comparative genomic hybridization microarray or sequencing; reagents for determining whether the TBX6 gene has a frameshift mutation include reagents used in sequencing; reagents for determining the haplotype of two SNP sites of rs3809624-rs3809627 in the TBX6 gene include reagents used in sequencing.

The reagents used in the QPCR include primers that amplify a nucleotide sequence of a length of 0.6 Mb between 29.5 Mb and 30.1 Mb in the chromosome 16p11.2 region, and the primer sequences are as follows: P1 site forward primer 5′-GGGGAAGGAACTTACATGAC-3′(SEQ ID NO: 1), P1 site reverse primer 5′-TCGTGTTTCCCTGTTGTACC-3′ (SEQ ID NO 2), PA site forward primer 5′ -GGTCTAAGCCACACACTAAC-3′(SEQ ID NO 3), PA site reverse primer 5′-TGAGTTTAGGGACCAATCTA-3′(SEQ ID NO 4), PB site forward primer 5′-GCTGCCAGTATGTGACCGAGA-3′(SEQ ID NO 5), PB site reverse primer 5′-GGGTGGAGGAGAGGATAGGG-3′ (SEQ ID NO: 6).

An agent for determining whether a TBX6 gene has a frameshift mutation includes a reagent used in sequencing, and the reagent includes a primer for amplifying a TBX6 gene, and the primer sequence is as follows: forward primer 5′-TAGGGAGAGGGCTCTGTTCTCATGG-3′ (SEQ ID NO 18), reverse primer 5′-GCGTCCCAGGGAGGCAACCG-3′ (SEQ ID NO: 19). The reagent may further comprise a primer for sequencing the nucleotide sequence of the TBX6 gene, the primer sequence being as follows: 5′-CTCGAAGGGGTCCGAGAGG-3′ (SEQ ID NO: 11), 5′-CTCCTTCCATAGCTCCCGGT-3′ (SEQ ID NO: 12), 5′ -GTTGCATACTGATCCCGAAT-3′(SEQ ID NO: 13), 5′-CTGCCCGAACTAGGTGTATG-3′ (SEQ ID NO: 14), 5′-AATGGCTTCCTAACAGATGAC-3′ (SEQ ID NO: 15), 5′-GAGCGGGAGGTTTGTGATG-3′ (SEQ ID NO: 16), 5′-GGCAGCTGGAAACACAGGT-3′ (SEQ ID NO: 17).

The reagent for determining the haplotype of two SNP sites of rs3809624-rs3809627 in the TBX6 gene includes an agent used in sequencing, and the reagent includes a primer for constructing a recombinant vector containing the TBX6 gene, and the primer sequence is as follows: T7 reverse primer 5′-TCGCCCTATAGTGAGTCGTATTACA-3′ (SEQ ID NO: 7), SP6 reverse primer 5′-GTATTCTATAGTGTCACCTAAATAG-3′ (SEQ ID NO: 8), CS forward primer 5′-GACTCACTATAGGGCGAGGGGAAGGGAGCGGGAGGTTTGTG-3′ (SEQ ID NO: 9), CS reverse primer 5′-GGTGACACTATAGAATACGCGCTGAGCCTGCCGGGAAGTGTAGT-3′ (SEQ ID NO: 10). The reagent may further comprise a primer for sequencing the nucleotide sequence of the TBX6 gene, the primer sequence being as follows: 5′ -CTCGAAGGGGTCCGAGAGG-3′ (SEQ ID NO: 11), 5′-CTCCTTCCATAGCTCCCGGT-3′ (SEQ ID NO: 12), 5′-GTTGCATACTGATCCCGAAT-3′(SEQ ID NO: 13), 5′-CTGCCCGAACTAGGTGTATG-3′(SEQ ID NO: 14), 5′-AATGGCTTCCTAACAGATGAC-3′(SEQ ID NO: 15), 5′-GAGCGGGAGGTTTGTGATG-3′(SEQ ID NO: 16), 5′-GGCAGCTGGAAACACAGGT-3′ (SEQ ID NO: 17).

Further, the diagnostic kit of the present application further includes an extraction reagent for genomic DNA. More preferably, the DNA extraction reagents include phenol, chloroform, isopropanol, and ethanol.

The naming rules for genetic mutations in this application are mainly based on the provisions of HGVS (Human Genome Variation Society). c denotes a coding DNA reference sequence (ie, a cDNA sequence), “del” denotes a deletion, and “ins” denotes an insertion.

Insertion refers to the phenomenon of one or more base additions compared to the reference sequence; represented by “ins”; named: “reference sequence prefix. nucleoside position ins”.

Deletion refers to the deletion of one or more nucleosides on the reference sequence of NCBI; the designation: “reference sequence prefix. nucleoside position del”.

As used herein, “c. 1248-1249insT” can also be expressed as “c. 1248-bit insertion T”, indicating that T is inserted between the 1248th and 1249th positions as compared with the reference sequence.

As used herein, “c. 263-264insC” may also be expressed as “c. 263 bit insertion C”, indicating that C is inserted between the 263th and 264th positions as compared with the reference sequence.

As used herein, “c. 697-698insG” may also be expressed as “c.697-bit insertion G”, indicating that G is inserted between the 697th and 698th positions as compared with the reference sequence.

As used herein, “c.1167-1168insC” may also be expressed as “c.1167-bit insertion C”, indicating that C is inserted between the 1167th and 1168th position as compared with the reference sequence.

As used herein, “c.1179-1180delAG” can also be expressed as “c.1179 double deletion AG”, indicating that the 1179th deletion of A and the 1180th deletion of G as compared with the reference sequence.

In a specific embodiment of the present application, the reference sequence for determining whether the TBX6 gene has a frameshift mutation refers to a cDNA sequence corresponding to the transcript of NM_004608.3 on NCBI, and the starting point of the cDNA is the first nucleotide of the initiation codon.

Advantages and benefit effects of the present application are as follows: this is the first time to reveal the genetic basis of human congenital scoliosis, and to diagnose congenital scoliosis through detecting deletions or nucleotide frameshift mutations in a gene and single nucleotide polymorphisms of a gene of an individual. A diagnostic kit for diagnosing congenital scoliosis prepared according to the above principle is sensitive and suitable for diagnosis of congenital scoliosis in early stage, and is capable of buying best time for early intervention for a patient.

### BRIEF DESCRIPTION OF THE DRAWINGS

FIG. 1 shows a chromosome 16p11.2 microdeletion analyzed and identified by a high-density oligonucleotide comparative genomic hybridization microarray;

FIG. 2 shows four single nucleotide insertions and a double-nucleotide deletion identified by Sanger sequencing;

FIG. 3 shows the construction schematic of a luciferase reporter system;

FIG. 4 shows effects of single nucleotide polymorphisms on gene expression detected by using a luciferase reporter gene.

### DETAILED DESCRIPTION OF THE INVENTION

The present application is further described below with reference to implementations. It should be understood that these embodiments are used for construing the present application only, but not limiting its scope. The experimental methods without specific conditions in the following embodiments are usually in accordance with the conventional conditions, or the manufacturer recommended conditions. Unless otherwise noted, the experimental methods used in the following examples are conventional methods. Unless otherwise noted, materials, reagents and the like used in the following examples are commercially available.

Supervision of the study: the applicant of the present disclosure ensures the integrity and accuracy of data and analysis. The present application has been approved by Ethics Committee of Chinese Academy of Medical Sciences & Peking Union Medical College, Institutes of Biomedical Sciences Fudan University, and Capital Institute of Pediatrics. All patients or family members thereof have provided handwritten informed consents to participate in the present application.

Information of the objects of the study: 237 Han Chinese who are not related to each other and suffer from congenital scoliosis were recruited. All patients are recruited from patients with congenital scoliosis confirmed by imaging examination in Chinese Academy of Medical Sciences & Peking Union Medical College Hospital from October 2010 to June 2014. Patients with known syndromes such as Alagille syndrome, Goldenhar's syndrome, hemifacial microsomia, Klippel-Feil syndrome, spondylocostal dysostosis and VACTERL syndrome are excluded. Two families which have 16p11.2 microdeletions but exhibit different phenotypes within the family are recruited from Affiliated Children's Hospital of Capital Institute of Pediatrics. Informed consents are obtained from all family members. A total of 166 healthy Han Chinese, who do not suffer from congenital scoliosis and do not have a genetic deficiency, are randomly selected as control.

Embodiment 1 Detection of Chromosome 16p11.2 Microdeletions

1. Blood Sampling and Extraction of Genomic DNA

5 ml morning fasting peripheral venous blood is collected from each object by using vacuum blood collection tubes containing EDTA anticoagulants, centrifugated at 3000 rpm/min for 10min, and then the plasma, the leucocyte layer and erythrocytes are separated. If possible, granulocytes can be further extracted by using a lymphocyte separation medium. If impossible, the leucocyte layer can be directly subpackaged in 2 ml freezing tubes and frozen for storage at −80° C. for subsequent use.

The genomic DNA is extracted by a phenol-chloroform method, purity of the DNA is determined by ultraviolet spectrophotometry (OD260/280 ratio), and concentration of the DNA is determined by OD₂₆₀, and after unified standardization the DNA is stored at −20° C. for subsequent use. Detailed steps are as follows:

(1) transferring the leucocyte suspension into a 5 ml centrifuge tube, adding a hemolytic reagent, centrifuging at 4000 rpm/min for 10 min after uniformly mixing by oscillating, then discarding the supernatant, and repeating the above process one time;

(2) adding 1 ml of extraction liquid into the precipitate, adding 8 μl of proteinase K after uniformly mixing, and keeping in water bath at 37° C. overnight;

(3) slightly cooling after taking out, then adding 1 ml of Tris-saturated phenol, inverting the tube up and down for 15 min to mix uniformly, and centrifuging at 4000 rpm/min for 10 min;

(4) carefully pipeting the supernatant, adding 60 μl of 3 M sodium acetate of pH 5.0, then adding isoamyl alcohol as equal volume of the supernatant, a flocculent white precipitate can be seen after gently shaking, and centrifuging at 10000 rpm/min for 2min;

(5) adding about lml of 75% alcohol into the precipitate, centrifuging at 8000 rpm/min for 2min, and discarding the supernatant;

(6) adding about 1 ml of anhydrous alcohol into precipitate, centrifuging at 8000 rpm/min for 2 min, and drying after discarding the supernatant;

(7) dissolving the dried product in 100 μl of TE buffer, determining the purity of the DNA by measuring OD₂₆₀₁₂₈₀ and OD₂₆₀₁₂₃₀ ratios, estimating the average size of the gDNA by 1% agarose gel electrophoresis, and storing the DNA at −20° C. after unified standardization; and

(8) the criteria for quality control of the DNA are: bands are obvious, with a length greater than 10 kb and without apparent degradations; the OD₂₆₀₁₂₈₀ is between 1.8 and 2.0, and the OD₂₆₀₁₂₃₀ is greater than 1.5, the criteria for detection are met.

2. Detection of Chromosome 16p11.2 Microdeletions in Small Samples

20 cases are selected from genomes of the 161 Han Chinese who are not related to each other and suffer from congenital scoliosis for oligonucleotide comparative genomic hybridization detection. DNA shearing, microarray processing and data analysis are performed according to the operational steps in the product instruction by using an Agilent's oligonucleotide comparative genomic hybridization microarray. Reference DNA is purchased from Promega.

3. Primary Screening in Large Samples by QPCR

Two detection sites (named as PA and PB) in the 16p11.2 microdeletion region and a reference site (named as P1) outside the 16p11.2 microdeletion region are selected. Different fragments are amplified by using P1 and PA or P1 and PB combination, and existing amount of the fragments is detected by a conventional QPCR method. The sequences of the primers used in the QPCR experiments are shown in Table 1:

TABLE 1
Sequences of the primers used in the detection
of 16p11.2 microdeletions

| Primer | Sequence |
|---|---|
| Primer 1-F | 5′-GGGGAAGGAACTTACATGAC-3′ |
| | (SEQ ID NO: 1) |
| Primer 1-R | 5′-TCGTGTTTCCCTGTTGTACC-3′ |
| | (SEQ ID NO: 2) |
| Primer A-F | 5′-GGTCTAAGCCACACACTAAC-3′ |
| | (SEQ ID NO: 3) |
| Primer A-R | 5′-TGAGTTTAGGGACCAATCTA-3′ |
| | (SEQ ID NO: 4) |
| Primer B-F | 5′-GCTGCCAGTATGTGACCGAGA-3′ |
| | (SEQ ID NO: 5) |
| Primer B-R | 5′-GGGTGGAGGAGAGGATAGGG-3′ |
| | (SEQ ID NO: 6) |

4.

Confirmation of the Results of the Primary Screening in Large Samples by QPCR

DNA samples with chromosome 16p11.2 microdeletions obtained from the primary screening in step (3) are confirmed according to the method described in step (1).

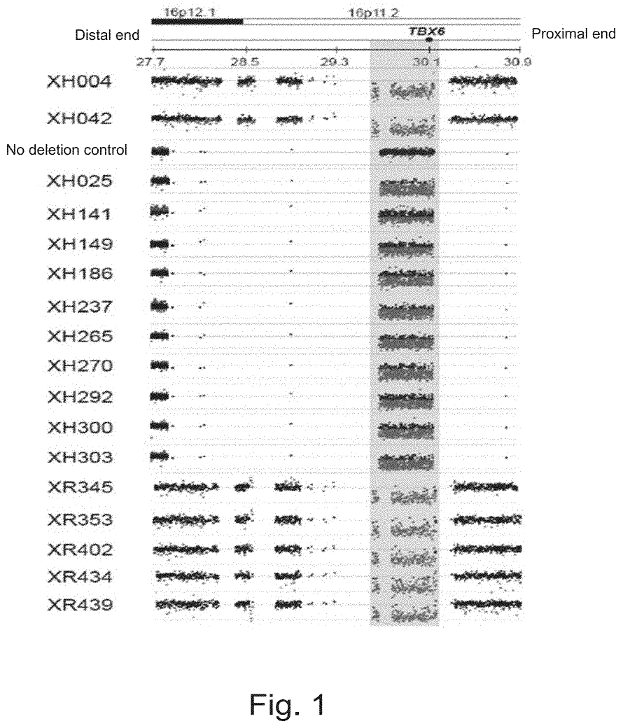

Results: genomes of 12 individuals of the 161 Han Chinese who are not related to each other and suffer from congenital scoliosis, have chromosome 16p11.2 microdeletions (the first 12 microdeletions shown in FIG. 1), and there is no chromosome 16p11.2 microdeletion in the healthy population by using the same method as described above to detect whether chromosome 16p11.2 microdeletions exist in the genomes of randomly selected 166 healthy Han Chinese (who do not suffer from congenital scoliosis) or not. The correlation between 16p11.2 microdeletions and CS is detected by a Fisher method, and P<0.0002.

Embodiment 2 Detection of Mutations in the TBX6 Gene

The common mechanism of nucleotide deletion related diseases is haploinsufficiency, such as the presence of copy of only one key gene is insufficient for an individual's physiological demands. Taking into account that haploinsufficiency of the TBX6 gene is a factor for the occurrence of CS, it may be considered that other factors which can lead to haploinsufficiency of the TBX6 gene may also be the factors leading to the occurrence of CS. Gene mutation is a common cause of diseases. Then, DNA of the TBX6 gene is sequenced to study whether the mutation exists or not.

1. Amplification of the Gene

The entire TBX6 gene coding regions and upstream regulatory regions of nearly 1 kb of 149 CS patients who are not related to each other and have no 16p11.2 microdeletion and 166 randomly selected normal individuals are amplified. The sequences of the primers are: forward primer 5′-TAGGGAGAGGGCTCTGTTCTCATGG-3′ (SEQ ID NO: 18); reverse primer 5′-GCGTCCCAGGGAGGCAACCG-3′ (SEQ ID NO: 19). The PCR amplification conditions are as follows:

$$\begin{matrix} 98 ° C . 1 min \\ \begin{matrix}98 ° C . 10 sec \\ 60 ° C . 20 sec\end{matrix} \} 35 cycles \\ 68 ° C . 4 min \\ 68 ° C . 10 min \\ 12 ° C . ∞\end{matrix}$$

PCR Amplification System(50 μl):

| | | |
|---|---|---|
| Water | 31.5 | μl |
| 10xLA PCR buffer II(Mg²⁺) | 5 | μl |
| dNTP(2.5 mM) | 8 | μl |
| forward primer(10 μM) | 2 | μl |
| reverse primer(10 μM) | 2 | μl |
| DMSO | 1 | μl |
| template (50 ng/μl) | 2 | μl |
| TaKaRa LA Taq(5 U/μl) | 0.5 | ul |

2. Sequencing

Determination of the TBX6 gene sequence is carried out by sequencing techniques which are well known to those skilled in the art. The sequencing primers are shown in Table 2:

TABLE 2
Sequences of the sequencing primers

| Primer | TBX6 gene fragment |
|---|---|
| 5′- | Upstream region |
| CTCGAAGGGGTCCGAGAGG-3 | |
| (SEQ ID NO: 11) | |
| 5′- | Upstream region and |
| CTCCTTCCATAGCTCCCGGT-3′ | exon 1 |
| (SEQ ID NO: 12) | |
| 5′- | |
| GTTGCATACTGATCCCGAAT-3′ | Exon 2 |
| (SEQ ID NO: 13) | |
| 5′- | |
| CTGCCCGAACTAGGTGTATG-3′ | Exon 3a |
| (SEQ ID NO: 14) | |
| 5′- | |
| AATGGCTTCCTAACAGATGAC- | Exons 3b, 4 and 5 |
| 3′ (SEQ ID NO: 15) | |
| 5′- | |
| GAGCGGGAGGTTTGTGATG-3′ | Exons 6, 7 and 8a |
| (SEQ ID NO: 16) | |
| 5′- | |
| GGCAGCTGGAAACACAGGT-3′ | Exon 8b and 3′- |
| (SEQ ID NO: 17) | UTR |

3. Results

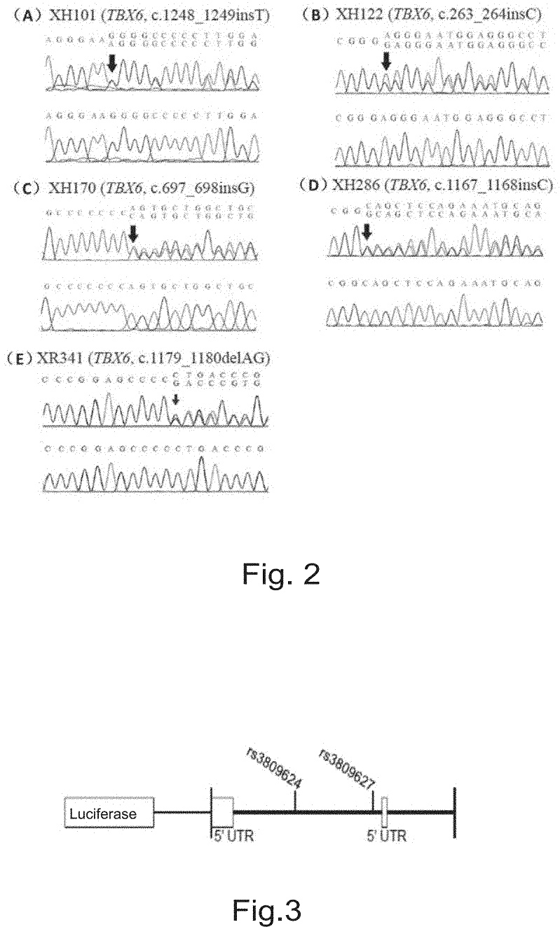

As shown in FIG. 2A-2D, frameshift mutations exist in the TBX6 genes of the genomes of 4 of the 149 Han Chinese who are not related to each other, have no 16p11.2 microdeletion and suffer from congenital scoliosis, the frameshift mutations are all caused by single nucleotide insertions, and the single nucleotide insertions are: c.1248-1249insT, c.263-264insC, c.697-698insG, c.1167-1168insC respectively. Whether the TBX6 gene frameshift mutations exist in the genomes of 166 healthy Han Chinese (without congenital scoliosis) who are randomly selected is detected by using the same method as described above, and no frameshift mutation is found in the healthy population. Besides, the TBX6 gene frameshift mutations are not found in 197 healthy Han Chinese in 1000 human genome project, either. The correlation between the TBX6 gene frameshift mutations and CS is detected by a Fisher method, and P<0.007.

Embodiment 3 Repeated Detection of Chromosome 16p11.2 Microdeletions and Mutations in the TBX6 Gene

1. Repeated Detection of Chromosome 16p11.2 Microdeletions

Object of study: 76 Han Chinese who are not related to each other and suffer from congenital scoliosis.

Method: the same as in Embodiment 1.

Result: the genomes of 5 of 76 Han Chinese who are not related to each other and suffer from congenital scoliosis have chromosome 16p11.2 microdeletions (the last five deletions in FIG. 1).

2. Repeated Detection of Mutations in the TBX6 Gene

Object of study: 71 Han Chinese who are not related to each other, have no chromosome 16p11.2 microdeletion and suffer from congenital scoliosis.

Method: the same as in Embodiment 2.

Result: 1 of 71 Han Chinese who are not related to each other, have no chromosome 16p11.2 microdeletion and suffer from congenital scoliosis has a double nucleotide deletion, i.e. deletion of AG at C1179 (as shown in FIG. 2E).

Embodiment 4 Detection of the Single Nucleotide Polymorphism of the TBX6 Gene

The phenotypes of the parents and siblings of members suffering from CS of two families SE1 and SE2 with 16p11.2 microdeletions are investigated, and it is found that some relatives of CS patients have 16p11.2 microdeletions but their phenotypes are normal, so the 16p11.2 microdeletions are not enough to cause the occurrence of CS, and other influence factors are involved, gene mutations are common factors leading to diseases, thus which genetic alteration of the TBX gene are present in the genomes of CS patients with 16p11.2 microdeletions is studied in the following.

Taking the members of the families SE1 and SE2 as objects of the study, the haplotypes of three SNP sites rs2289292-rs3809624-rs3809627 in the TBX6 gene on a chromosome without 16p11.2 microdeletion are detected. The specific operation is: using a ClonExpress One Step Cloning Kit (Vazyme) to detect the haplotypes of common TBX6 gene variants; using a pGEM-T vector as a template for the amplification of the vector to amplify the vector and inserted DNA fragments respectively, connecting; transforming the recombinant vector into competent cells of Escherichia coli; selecting clones, and detecting sequence by using sanger sequencing. The primer sequences used in the experiments are shown in Table 3:

TABLE 3
Primer sequences used in haplotype detection

| Primer | Sequence |
|---|---|
| T7 reverse | 5′-TCGCCCTATAGTGAGTCGTATTACA-3′ |
| primer | (SEQ ID NO: 7) |
| SP6 reverse | 5′-GTATTCTATAGTGTCACCTAAATAG-3′ |
| primer | (SEQ ID NO: 8) |
| CS forward | 5′-GACTCACTATAGGGCGAGGGGAAGGGAGCGGGA |
| primer | GGTTTGTG-3′ |
| | (SEQ ID NO: 9) |
| CS reverse | 5′- |
| primer | GGTGACACTATAGAATACGCGCTGAGCCTGCCGGGA |
| | AGTGTAGT-3′ (SEQ ID NO: 10) |

Result: the sequencing results show that the haplotypes of the three SNP sites rs2289292-rs3809624-rs3809627 of the members with CS in the families SE1 and SE2 are T-C-A, but the haplotypes of their parents or siblings are not T-C-A, thus the presence of haplotype T-C-A increases the chance of CS disease.

The haplotypes of the three SNP sites rs2289292-rs3809624-rs3809627 of the TBX6 gene on a normal chromosome of the 22 individuals selected in Embodiments 1 to 3 with genetically defective genomes are detected and it is found that the haplotypes of the three SNP sites rs2289292-rs3809624-rs3809627 of 22 patients are all T-C-A.

Embodiment 5 Detection of the Effects of Single Nucleotide Site on the Expression of the TBX6 Gene

Experimental Steps:

(1) Amplifying a 1120 bp DNA fragment of an upstream regulatory element of the TBX 6 gene, and constructing a normal DNA fragment, a DNA fragment with only rs3809624 site mutated to C, a DNA fragment with only rs3809627 site mutated to A and a DNA fragment with rs3809624 site and rs3809627 site mutated at the same time onto a pGL3-Basic vector respectively (construction mode is shown in FIG. 3).

(2) Transfecting recombinant vectors into HEK293T, HepG2, Hela cells cultured in vitro.

(3) After transfection for a certain period of time, lysing the cells and obtaining supernatants to detect the activity of luciferase by using a Dual-Luciferase Reporter Gene

Assay System.

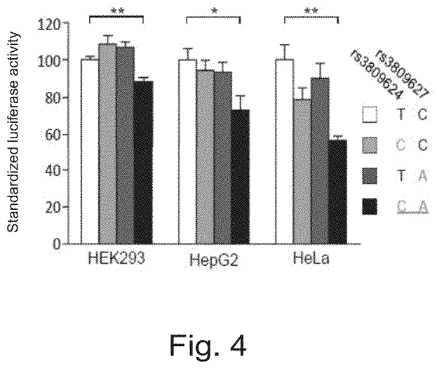

The results are shown in FIG. 4, in the three types of cells, the mutations of only rs3809624 or rs3809627 sites cannot affect the expression of a reporter gene, only both of the two sites mutate, the expression of the reporter gene is suppressed, so the double mutations of the rs3809624 and rs3809627 sites exist at the same time on the TBX6 gene of CS patients cause the down-regulated TBX6 gene expression.

While embodiments of the present application have been shown and described, it would be understood by those of skilled in the art that various changes, modifications, substitutions and alterations can be made in these embodiments without departing from the principles and objects of the present application, the scope of the present application is defined by the claims and their equivalents.

What is claimed is:

1. A method for diagnosing congenital scoliosis of an individual, comprising:
detecting whether a chromosome 16p11.2 region has a nucleotide sequence microdeletion of 0.6 Mb in length, or detecting whether a TBX6 gene has a frameshift mutation; and
detecting a haplotype of two SNP sites of rs3809624-rs3809627 in the TBX6 gene located on another homologous chromosome;
if a microdeletion of 0.6 Mb in length exists in the chromosome 16p11.2 region, meanwhile the haplotype of two SNP sites of rs3809624-rs3809627 in the TBX6 gene located on the another homologous chromosome without 16p11.2 microdeletion is C-A, the individual is diagnosed as a patient with congenital scoliosis;
if the TBX6 gene in the chromosome 16p11.2 region has the following single nucleotide insertion and double nucleotide deletion: one or more of nucleotide shift mutations caused by c.1248-1249insT, c.263-264insC, c.697-698insG, c.1167-1168insC, c.1179-1180delAG, and the haplotype of two SNP sites of rs3809624-rs3809627 in the TBX6 gene located on the homologous chromosome without frameshift mutations is C-A, then the individual is diagnosed as a patient with congenital scoliosis.

2. The method according to claim 1, wherein the method further comprises the following steps:
(1) obtaining a biological sample containing the genomic DNA of the subject; and
(2) extracting genomic DNA in the biological sample.

3. The method according to claim 1, wherein the detection of whether a microdeletion of 0.6 Mb in length exists in the chromosome 16p11.2 region is implemented by using QPCR, high-density oligonucleotide comparative genomic hybridization microarray or sequencing; the detection of whether a TBX6 gene has a frameshift mutation is implemented by sequencing; and the detection of the haplotype of two SNP sites of rs3809624-rs3809627 in the TBX6 gene is implemented by sequencing.

4. The method according to claim 3, wherein the use of QPCR to detect whether a microdeletion of 0.6 Mb in length exists in the chromosome 16p11.2 region is implemented by the use of primers for amplifying a nucleotide sequence having a length of 0.6 Mb located between 29.5 Mb and 30.1 Mb in the chromosome 16p11.2 region, and the sequences of the primers are as follows: P1 site forward primer5′-GGGGAAGGAACTTACATGAC-3′ (SEQ ID NO: 1), P1 site reverse primer 5′-TCGTGTTTCCCTGTTGTACC-3′ (SEQ ID NO: 2), PA site forward primer 5′ -GGTCTAAGCCACACACTAAC-3′ (SEQ ID NO: 3), PA site reverse primer 5′-TGAGTTTAGGGACCAATCTA-3′ (SEQ ID NO: 4), PB site forward primer 5′-GCTGCCAGTATGTGACCGAGA-3′ (SEQ ID NO: 5), PB site reverse primer 5′-GGGTGGAGGAGAGGATAGGG-3′ (SEQ ID NO: 6).

5. The method according to claim3, wherein the use of sequencing to detect whether a TBX6 gene has a frameshift mutation requires amplification primers and sequencing primers, the amplification primers are as follows: forward primer 5′-TAGGGAGAGGGCTCTGTTCTCATGG-3′ (SEQ ID NO: 18); reverse primer 5′-GCGTCCCAGGGAGGCAACCG-3′ (SEQ ID NO: 19); the sequencing primers are as follows: 5′-CTCGAAGGGGTCCGAGAGG-3′ (SEQ ID NO: 11), 5′-CTCCTTCCATAGCTCCCGGT-3′ (SEQ ID NO: 12), 5′-GTTGCATACTGATCCCGAAT-3′(SEQ ID NO: 13), 5′-CTGCCCGAACTAGGTGTATG-3′ (SEQ ID NO: 14), 5′-AATGGCTTCCTAACAGATGAC-3′ (SEQ ID NO: 15), 5′-GAGCGGGAGGTTTGTGATG-3′ (SEQ ID NO: 16), 5′-GGCAGCTGGAAACACAGGT-3′ (SEQ ID NO: 17).

6. The method according to claim 2, wherein the biological sample comprises tissue and body fluids.

7. The method according to claim 6, wherein the biological sample is a body fluid, and the body fluid includes blood, plasma, saliva, urine, and amniotic fluid.

8. The method according to claim 7, wherein the biological sample is blood.

9. A diagnostic kit for congenital scoliosis, comprising: an agent for determining whether a chromosome 16p11.2 region has a nucleotide sequence microdeletion of 0.6 Mb in length, or determining whether a TBX6 gene has a frameshift mutation, and determining the haplotype of two SNP sites of rs3809624-rs3809627 in the TBX6 gene located on another homologous chromosome;
the frameshift mutation of the TBX6 gene is selected from the following single nucleotide insertions and dinucleotide deletions: one or more of nucleotide shift mutations caused by c.1248-1249insT, c.263-264insC, c.697-698insG, c.1167-1168insC, c.1179-1180delAG.

10. The diagnostic kit for congenital scoliosis according to claim 9, wherein the reagents for determining whether a chromosome 16p11.2 region has a nucleotide sequence microdeletion of 0.6 Mb in length include the reagents used in QPCR, high-density oligonucleotide comparative genomic hybridization microarray or sequencing; reagents for determining whether the TBX6 gene has a frameshift mutation include reagents used in sequencing; reagents for determining the haplotype of two SNP sites of rs3809624-rs3809627 in the TBX6 gene include reagents used in sequencing.

11. The diagnostic kit for congenital scoliosis according to claim 10, wherein the reagents used in the QPCR include primers that amplify a nucleotide sequence of a length of 0.6 Mb between 29.5 Mb and 30.1 Mb in the chromosome 16p11.2 region, and the primer sequences are as follows: P1 site forward primer 5′-GGGGAAGGAACTTACATGAC-3′ (SEQ ID NO: 1), P1 site reverse primer 5′-TCGTGTTTCCCTGTTGTACC-3′ (SEQ ID NO: 2), PA site forward primer 5′-GGTCTAAGCCACACACTAAC-3′ (SEQ ID NO: 3), PA site reverse primer 5′-TGAGTTTAGGGACCAATCTA-3′ (SEQ ID NO: 4), PB site forward primer 5′-GCTGCCAGTATGTGACCGAGA-3′ (SEQ ID NO: 5), PB site reverse primer 5′-GGGTGGAGGAGAGGATAGGG-3′ (SEQ ID NO: 6).

12. The diagnostic kit for congenital scoliosis according to claim 10, wherein the reagents used in the sequencing include sequencing primers, the sequencing primer sequences are as follows: 5′-CTCGAAGGGGTCCGAGAGG-3′ (SEQ ID NO: 11), 5′-CTCCTTCCATAGCTCCCGGT-3′ (SEQ ID NO: 12), 5′-GTTGCATACTGATCCCGAAT-3′ (SEQ ID NO: 13), 5′-CTGCCCGAACTAGGTGTATG-3′ (SEQ ID NO: 14), 5′-AATGGCTTCCTAACAGATGAC-3′ (SEQ ID NO: 15), 5′-GAGCGGGAGGTTTGTGATG-3′(SEQ ID NO: 16), 5′-GGCAGCTGGAAACACAGGT-3′ (SEQ ID NO: 17).
